(19) United States
(12) Patent Application Publication
(10) Pub. No.: US 2015/0303397 A1
(43) Pub. Date: Oct. 22, 2015

(54) INORGANIC BARRIER LAYERS

(71) Applicant: MERCK PATENT GMBH, Darmstadt (DE)

(72) Inventors: Junyou Pan, Frankfurt Am Main (DE); Andreas KLYSZCZ, Darmstadt (DE)

(73) Assignee: MERCK PATENT GMBH, Darmstadt (DE)

(21) Appl. No.: 14/652,310

(22) PCT Filed: Nov. 19, 2013
(86) PCT No.: PCT/EP2013/003482

§ 371 Date: Jun. 15, 2015

(30) Foreign Application Priority Data
Dec. 17, 2012 (EP) 12008383.7

(51) Int. Cl. H01L 51/50; H01L 51/56; A61N 5/06; H01L 51/52

(52) U.S. Cl. CPC H01L 51/5096; H01L 51/5206; H01L 51/5221; H01L 51/5012; H01L 51/5072; H01L 51/5092; H01L 51/504; H01L 51/56; A61N 5/0616; H01L 2251/303; H01L 51/005

(57) ABSTRACT

An organic electroluminescent device the emits light in the UV and blue region of the visible spectrum, the device comprising an anode, a cathode, at least one emission layer and at least one exciton-blocking layer (ExBL) between an emission layer and anode, wherein the exciton-blocking layer includes an inorganic material having a wide band gap and the use of the device in phototherapy.

The present invention relates to organic electroluminescent devices comprising inorganic blocking layers and to the use thereof.

The structure of organic electroluminescent devices (for example organic light-emitting diodes—OLEDs) in which organic semiconductors are employed as functional materials is described, for example, in U.S. Pat. No. 4,539,507, U.S. Pat. No. 5,151,629, EP 0676461 and WO 98/27136. The emitting materials employed here, besides fluorescent emitters, are increasingly organometallic complexes which exhibit phosphorescence (M. A. Baldo et al., Appl. Phys. Lett. 1999, 75, 4-6). For quantum-mechanical reasons, an up to four-fold energy and power efficiency is possible using organometallic compounds as phosphorescence emitters. In general, there is still a need for improvement, in particular with respect to efficiency, operating voltage and lifetime, both in the case of OLEDs which exhibit singlet emission and also in the case of OLEDs which exhibit triplet emission. This applies, in particular, to OLEDs which emit in the relatively short-wave region, i.e. green and in particular blue. The efficiency of OLEDs can be significantly increased by the use of a blocking layer, in particular an exciton-blocking layer. An organic exciton-blocking material is usually employed here. Since organic compounds have a large exchange energy of (about 0.7-1.0 eV, the energy gap between singlet and triplet state in organic compounds is large and is about 0.5 eV or more (Kohler & Bässler, Materials Science and Engineering R 66 (2009) 71-109). However, the organic blocking materials can only be employed to a limited extent in OLEDs which comprise triplet emitters, in particular if the OLED emits blue light. This is due to the requirements made of exciton-blocking materials in blue-emitting triplet OLEDs. The blocking materials must in this case have a very large band gap which are at least 0.5 eV larger than the corresponding blocking materials in singlet OLEDs. To date, the choice of exciton-blocking materials, in particular for triplet blue OLEDs, is very limited. There is therefore still a great need to provide such materials for use in OLEDs.

The use of such materials in blue-electroluminescent, phosphorescent devices would result in significant increases in efficiency.

Furthermore, there are many applications which require light or radiation having even shorter wavelengths, for example 280 to 400 nm are necessary for cell imaging or for biosensors in the life science and medicine area. Furthermore, in the electronics industry 300 to400 nm are required for solid-state lighting and 300 to 365 nm for the curing of polymers and printing ink, etc. Phototherapeutic applications in the medical or cosmetic area are also of major importance. Many undesired skin changes and skin diseases can be treated by means of phototherapy. Wavelengths in the region of ultraviolet (UV) radiation are often required for this purpose. An example thereof is the phototherapeutic treatment of the skin of psoriatic patients, for which a radiation source which emits UV radiation of a wavelength of 311 nm is typically employed.

Mercury, deuterium, excimer and xenon lamps are typical, conventional UV radiation sources. However, they are bulky and some contain toxic substances which cause soiling and may represent health risks. Conventional lamps therefore have disadvantages with respect to safety, usability, handling and portability, which in turn results in limited areas of application. In addition, UV-LEDs are also commercially available, but practical LED arrays below 365 nm are very restricted. In addition, LEDs have the disadvantage that they are point emitters, which require relatively thick and rigid devices. Another class of radiation sources or light sources are organic electroluminescent devices (for example OLED-organic light-emitting diodes or OLEC-organic light-emitting electrochemical cell). These are area emitters which also allow the production of flexible devices. Owing to their efficiency and the simple and space-saving structure, these devices are also particularly suitable for many applications.

However, there continues to be a great need to improve organic electro-luminescent devices, in particular also those which emit radiation in the UV region, with respect to efficiency, lifetime, and radiation intensity.

Chao et al., reports (Adv. Mater. 17[8], 992-996. 2005.) UV OLEDs based on fluorene polymers having an electroluminescence emission wavelength greater than 360 nm;

Wong et al., reports (Org. Lett. 7[23], 5131-5134. 2005) UV OLEDs based on spirobifluorene polymer having electroluminescence emission wavelength around or greater than 360 nm;

Zhou et al., reports (Macromolecules 2007, 40 (9), 3015-3020) UV OLEDs comprising emitting polymers based on fluorene and tetraphenylsilane derivatives having electroluminescence of an emission wavelength at 350 nm;

Shinar et al., reports (Applied Surface Science 2007, 254 (3), 749-756) UV OLEDs using Bu-PBD as emitter having an electroluminescence emission wavelength of 350 nm;

Burrows reports (Applied Physics Letters 2006, 88 (18), 183503) an OLED comprising 4,4′-bis (diphenyiphosphine oxide) biphenyl as emitter; the device emits at 337 nm.

Sharma et al., reports (Applied Physics Letters 2006, 88 (14), 143511-143513) a UV-OLED which emits at 357 nm. The emitter used is based on polysilane.

However, the efficiency of the said devices which emit UV radiation in the UV region is still unsatisfactory. There is therefore a need to develop efficient organic electroluminescent devices or novel device structures which emit radiation in the UV region, in particular in the lower UV-A region (315 to 380 nm) and in the UV-B region (280 to 315 nm).

The object of the present invention is therefore the elimination of the said disadvantages of the prior art by the provision of organic electroluminescent devices which efficiently emit light or radiation, in particular in the blue region of the visible spectrum and in the UV region.

Surprisingly, it has been found that the use of inorganic materials having a large band gap, which are usually used as insulators or dielectric materials, in an exciton-blocking layer in organic electroluminescent devices result in significant improvements of the devices. The present invention therefore relates to organic electroluminescent devices which comprise compounds of this type and to processes for the production of the devices.

The present invention relates to an organic electroluminescent device comprising an anode, a cathode, at least one emission layer and at least one exciton-blocking layer (ExBL) between an emission layer and anode, characterised in that the exciton-blocking layer comprises an inorganic material.

The inorganic material is responsible for blocking the excitons. An ExBL relates to a layer which is applied directly to an adjacent layer which comprises or generates excitons (typically an emitting layer in a multilayered OLED), characterised in that the ExBL prevents the diffusion from the layer which comprises the excitons through the blocking layer into the next layer. In other words, it is possible to keep excitons in a certain layer, for example in the emission layer. The efficiency of electroluminescent devices can thus be increased.

The ExBL normally has a higher energy of the same type of excitons compared with the emitting layer. For a triplet OLED, this means that the ExBL has a higher triplet level than the emission layer. For a singlet OLED, this means that the singlet level of the ExBL is higher than that of the emission layer.

Inorganic materials have a high dielectric constant and band structure. The exchange energy of inorganic materials is zero (Kohler & Bassler, Materials Science and Engineering R 66 (2009) 71-109). There is no difference here between singlet and triplet excited states and the energy of the first excited states is determined by the band gap between conduction band and valence band (called band gap below).

The band gap of inorganic materials can be determined by both spectroscopic methods, by measurement of the conductivity or by means of other methods which are well known to the person skilled in the art. It can also be determined, for example, from absorption and reflection spectra (Aleshin et al., Soy. Phys.—Solid State, 10, 2282 (1969)), from photoconductivity measurements (Ali et al., Phys. Status Solidi, 28, 193 (1968)) and from measurements of the thermal activation energy of the electrical conductivity (Altpert et al., Solid State Commun., 5, 391 (1967)). In this connection, measurement of the absorption edge is the most frequently used. Furthermore, comprehensive reviews exist in which band gaps of inorganic materials are indicated (Strehlow et al., in J. Phys. Chem. Ref. Data Vol2, p163 (1973), and Pelatt et al., in J. Am. Chem. Soc. Vo1133, p16852 (2011). Unless indicated otherwise, the band gaps used in the present disclosure have been taken from the literature cited.

In a preferred embodiment of the present invention, the inorganic material in the exciton-blocking layer has a band gap of at least 3.5 eV, preferably at least 3.8 eV, very preferably at least 4 eV and very particularly preferably at least 4.2 eV.

The inorganic material in the blocking layer of the device is furthermore preferably a metal chalcogenide.

Metal chalcogenides here are intended to be taken to mean chemical compounds which are formed from one or more chalcogen elements (oxygen, sulfur, selenium and tellurium) as formal anions with metals or more strongly electropositive elements (such as arsenic, germanium, phosphorus, antimony, lead, boron, aluminium, gallium, indium, titanium or sodium) as formal cations. Preferred metal chalcogenides are the metal oxides and metal sulfides, very preferably the metal oxides.

The inorganic material of the blocking layer is very preferably selected from the group consisting of the compounds BaO, MgO, Al₂O₃, SrO, HfO₂, ZrO₂, GeO₂, Ga₂O₃, Ta₂O₅ or a mixture of the said compounds. Very particular preference is given here to the compounds HfO₂, ZrO₂

In an embodiment, the blocking layer comprises an organic compounds and an inorganic compound, where the blocking layer comprises 15% by weight to 100% by weight, preferably 20% by weight to 100% by weight, very preferably 40% by weight to 100% by weight, very particularly preferably 50% by weight to 100% by weight and especially preferably 60% by weight to 100% by weight (based on the entire blocking layer) of the inorganic compound.

In a particularly preferred embodiment of the present invention, the the blocking layer consists exclusively of the inorganic material having a large band gap or of a mixture of these inorganic materials. The blocking layer very particularly preferably consists of one of the materials BaO, MgO, Al₂O₃SrO, HfO₂, ZrO₂, GeO₂, Ga₂O₃, Ta₂O₅ or of a mixture of the said materials, it preferably consists of HfO₂ or ZrO₂, very preferably of HfO₂.

The device according to the invention can emit in a broad wavelength range. The device according to the invention preferably emits radiation in the range from 280 to 380 nm and preferably in the range from 280 to 360 nm.

For the purposes of the present invention, the at least one emission layer of the device according to the invention preferably comprises at least one emitter which emits radiation having a wavelength in the range from 280 to 380 nm and preferably in the range from 280 to 360 nm.

The person skilled in the art will be able to make a selection here from a multiplicity of emitters which are known in the from the prior art. Some emitters which can be employed for the said purpose are indicated by way of example below.

It is especially preferred if the at least one emission layer of the device according to the invention comprises at least one compound of the general formula (1) as emitter or as host.

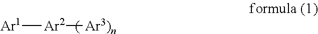

and where the following applies to the symbols and indices used:
Ar¹, Ar² and Ar³ are, identically or differently, five- or six-membered aromatic and/or heteroaromatic rings, which may in each case be substituted by one or more radicals R¹, which may be independent of one another;
n
   is 0 or 1;
X
   is on each occurrence, identically or differently, CR¹ or N;
Q
   is on each occurrence, identically or differently, X═X, NR¹, O, S, Se, preferably, X═X, NR¹ and S and very preferably X═X and NR¹.
R¹
   is, identically or differently on each occurrence, H, D, F, Cl, N(R²)₂, CN, Si(R²)₃, B(OR²)₂, C(═O)R², P(═O)(R²)₂, P(R²)₂, S(═O)R², a straight-chain alkyl, alkoxy or thioalkoxy group having 1 to 40 C atoms or a branched or cyclic alkyl, alkoxy, alkylalkoxy or thioalkoxy group having 3 to 40 C atoms, each of which may be substituted by one or more radicals R², where one or more non-adjacent CH₂ groups which is not bonded directly to Ar¹, Ar² or Ar³ in formula (1) may be replaced by R²C═CR², C≡C, Si(R²)₂, Ge(R²)₂, Sn(R²)₂, C═O, C═S, C═Se, C═NR², P(═O)(R²), SO, SO₂, NR², O, S or CONR² and where one or more H atoms may be replaced by D, F, Cl, or CN, or an aromatic or heteroaromatic ring having 5 to 18 aromatic ring atoms, which may in each case be substituted by one or more radicals R², or an aryloxy, arylalkoxy or heteroaryloxy group having 5 to 60 aromatic ring atoms, which may be substituted by one or more radicals R², or a combination of two or more of these groups, two or more substituents R¹ may also form a non-aromatic ring system with one another here;
R²
   is, identically or differently on each occurrence, H, D, F, Cl, N(R³)₂, CN, Si(R³)₃, B(OR³)₂, C(═O)R³, P(═O)(R³)₂, S(═O)R³, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which may be substituted by one or more radicals R³, where one or more non-adjacent CH₂ groups may be replaced by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³ and where one or more H atoms may be replaced by D, F, Cl or CN, or an aromatic or heteroaromatic ring having 5 to 18 aromatic ring atoms, which may in each case be substituted by one or more radicals R³, or a combination of two or more of these groups; two or more adjacent radicals R² may form a non-aromatic ring system with one another here;
R³
   is, identically or differently on each occurrence, H, D, F or an aliphatic, aromatic and/or heteroaromatic hydrocarbon radical having 1 to 18 C atoms, in which, in addition, one or more H atoms may be replaced by F; two or more substituents R³ may also form a non-aromatic mono- or polycyclic, aliphatic ring system with one another here;

with the proviso that the compound of the formula (1) does not contain any condensed aromatic or condensed heteroaromatic ring systems, and the number of electron in a fully conjugated moiety is not more than 18.

The compounds of the general formula (1) and the preparation thereof are disclosed, for example, in EP12007076.8. The preferred embodiments disclosed therein also represent preferred emitters for the present invention, which are employed as UV emitters in the emission layer. Some selected examples of especially preferred UV emitters are summarised in the following overview.

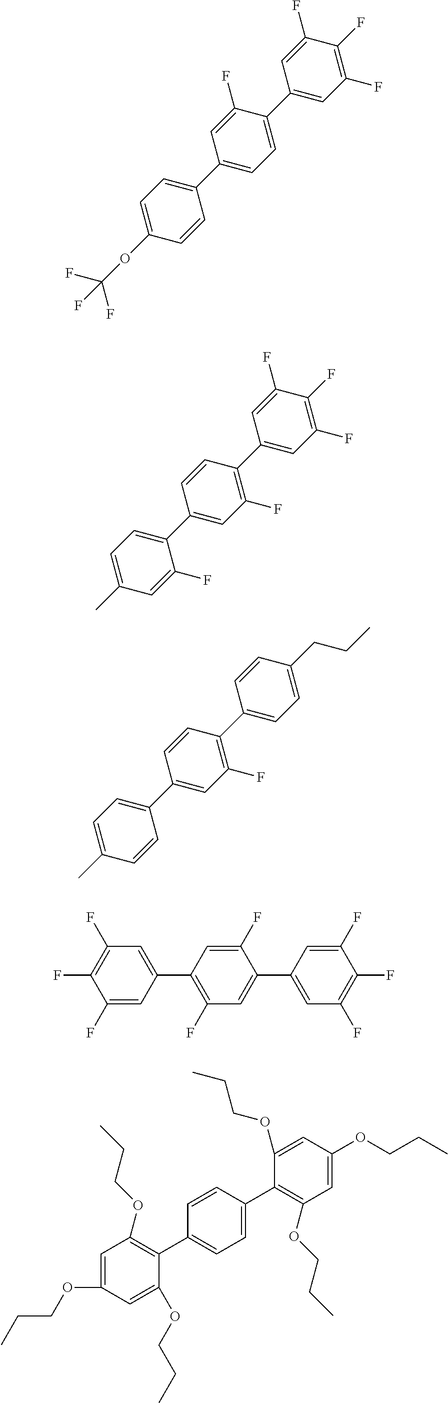

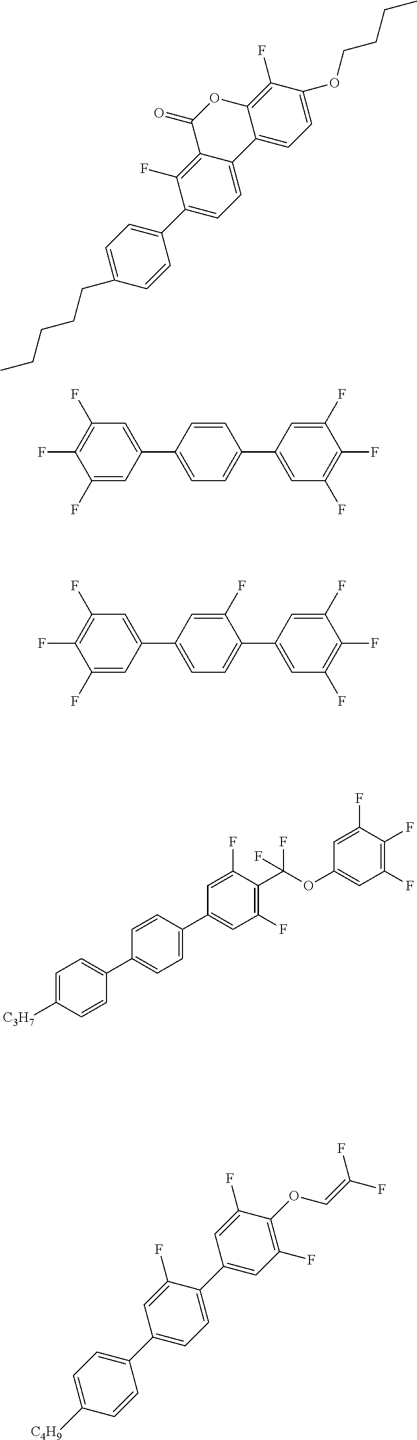

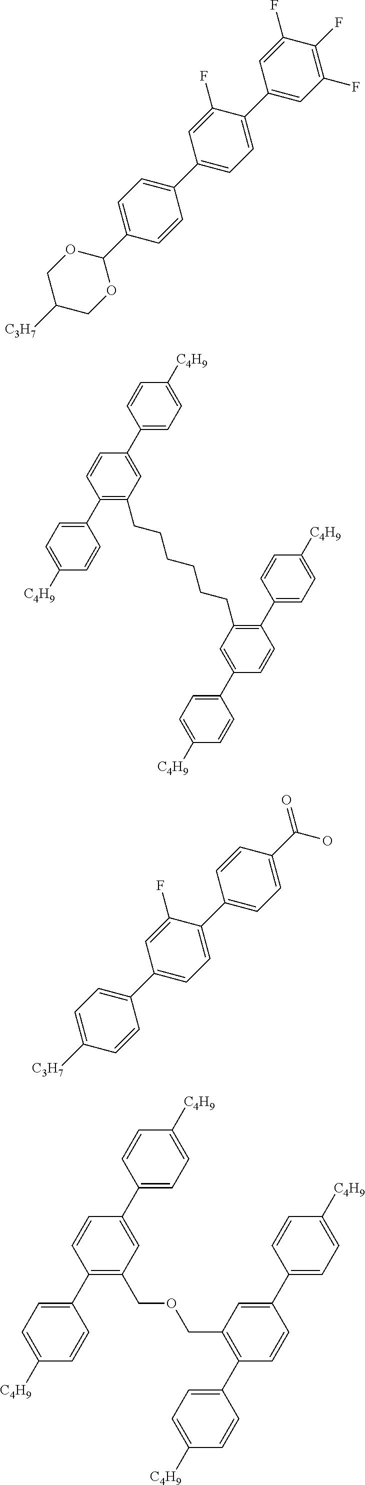

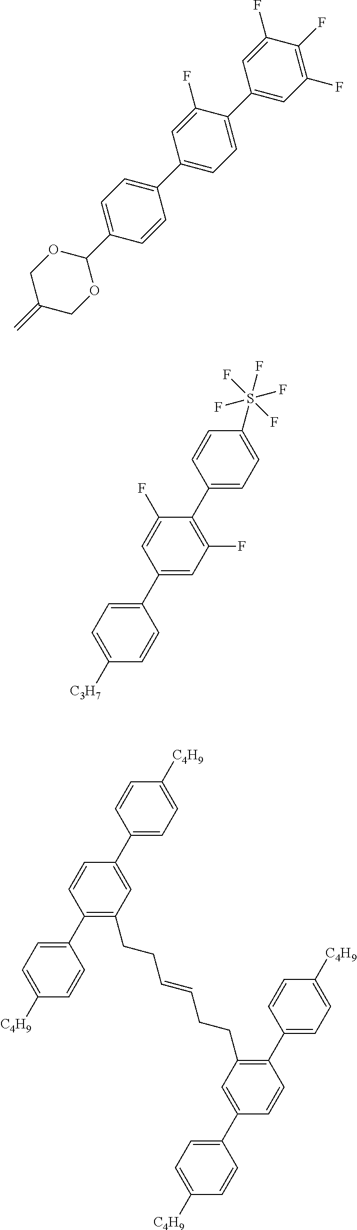

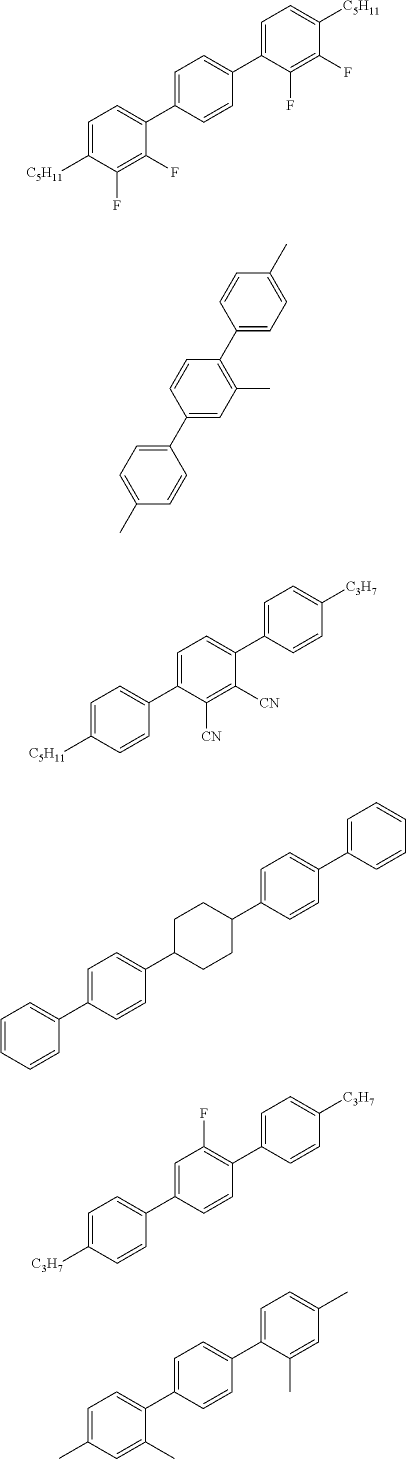

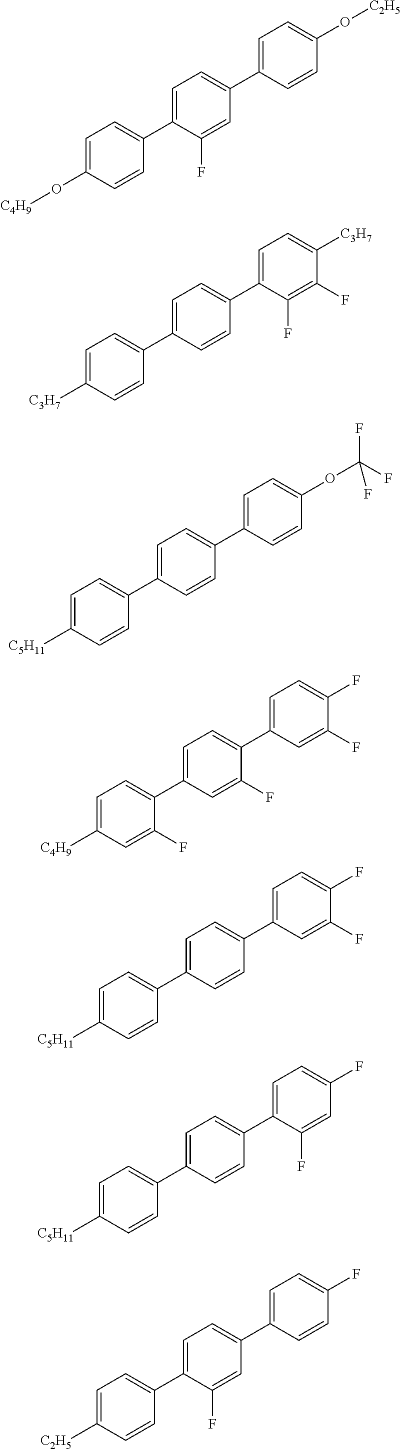

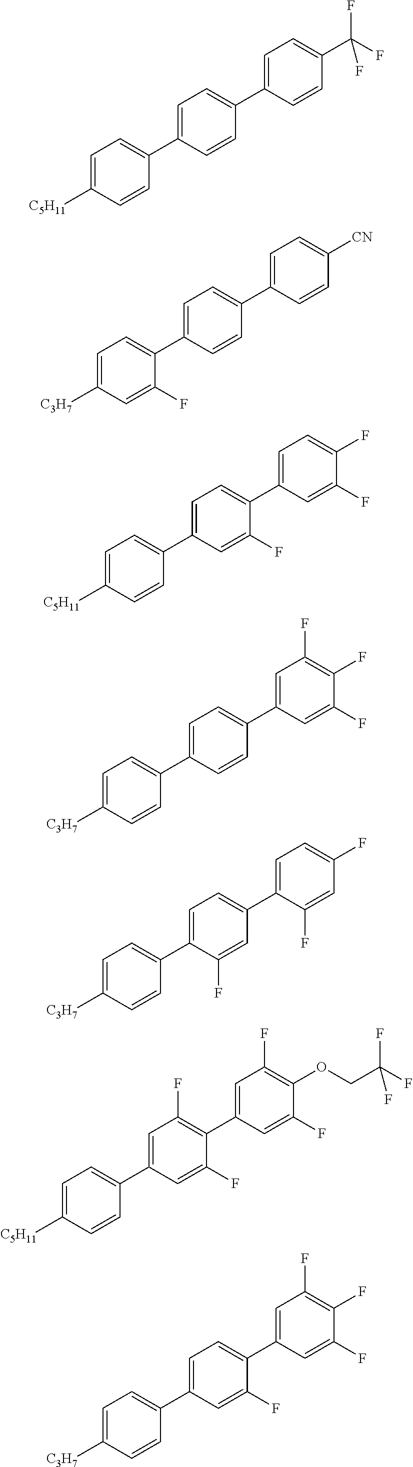

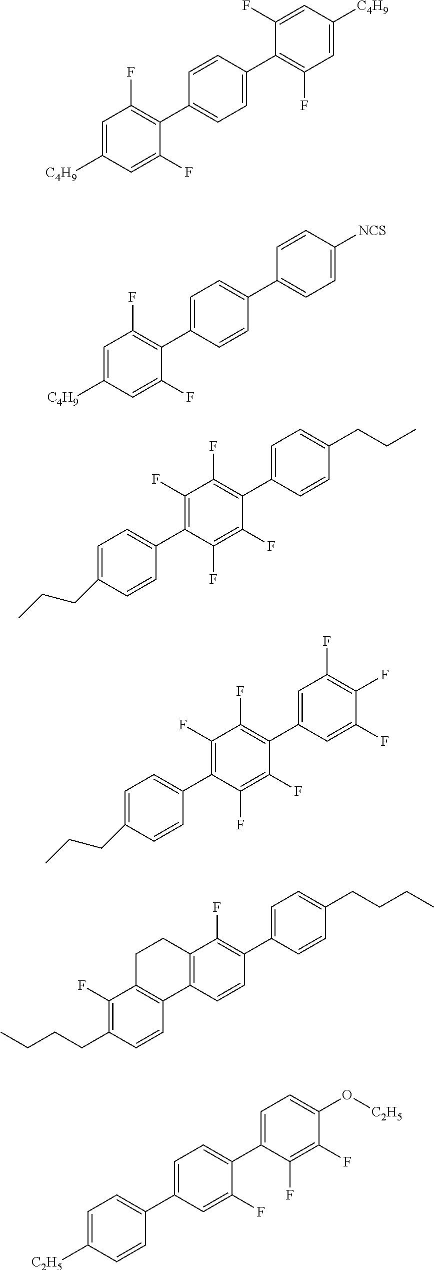

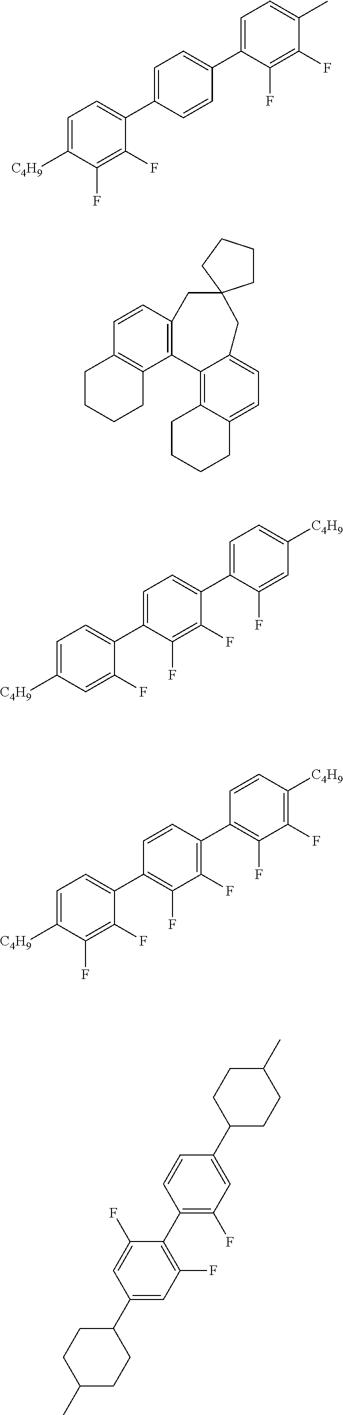

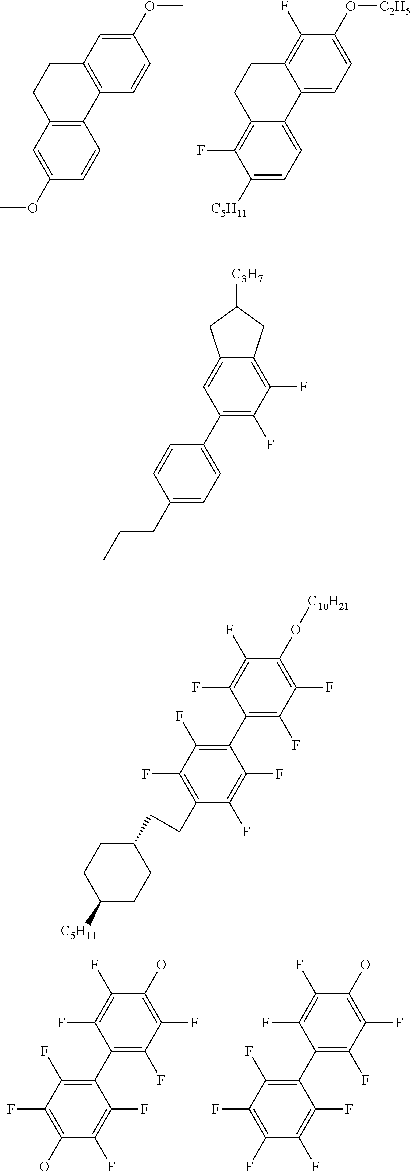

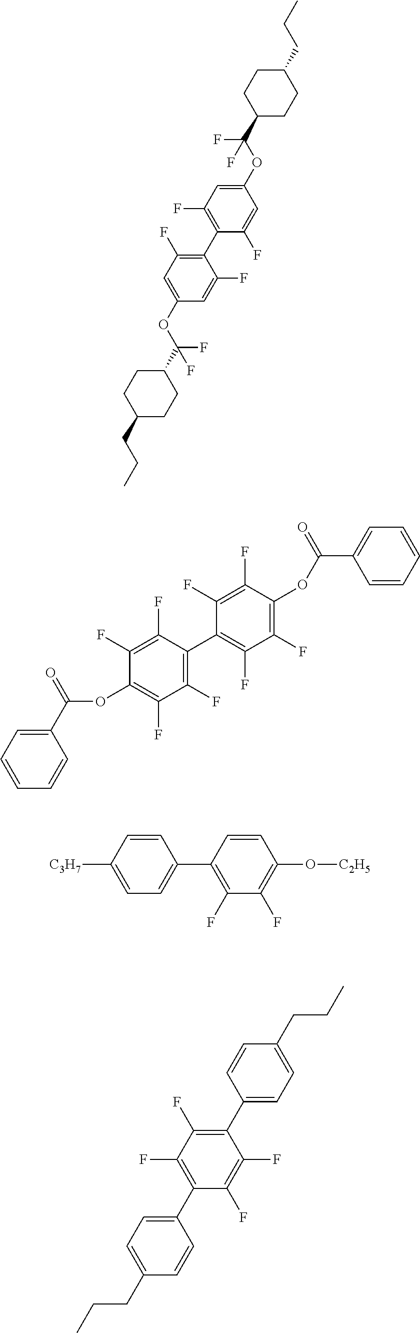

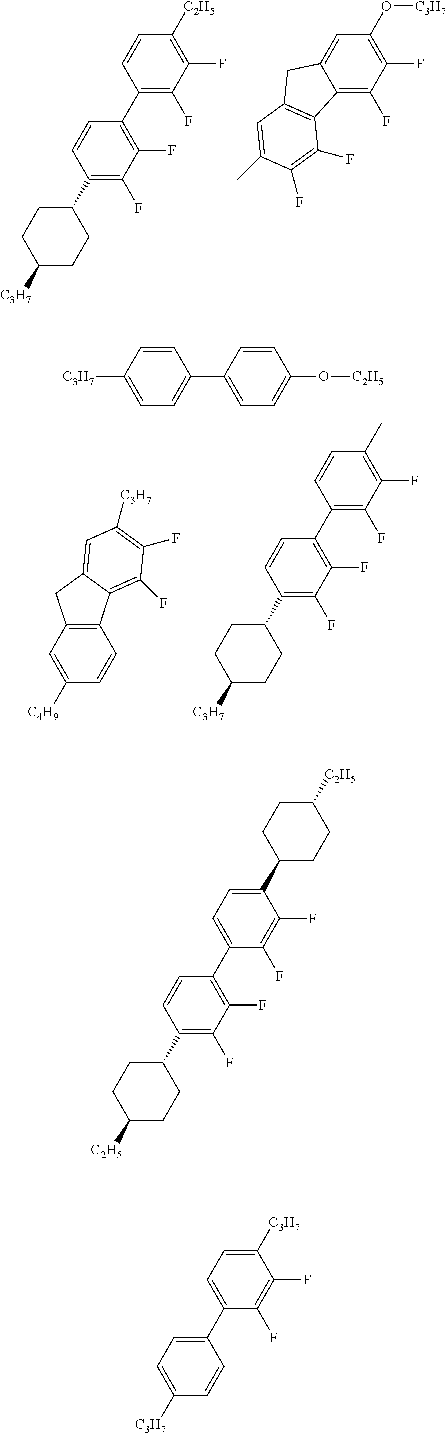

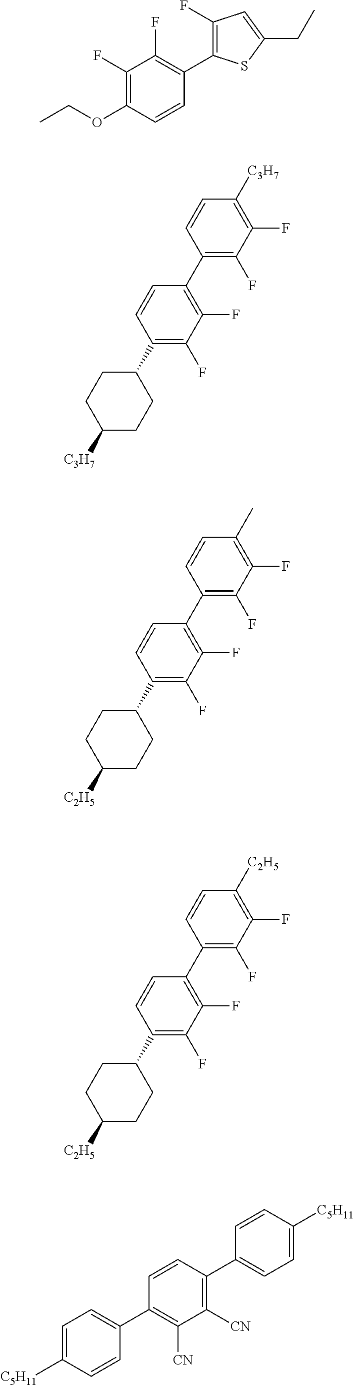

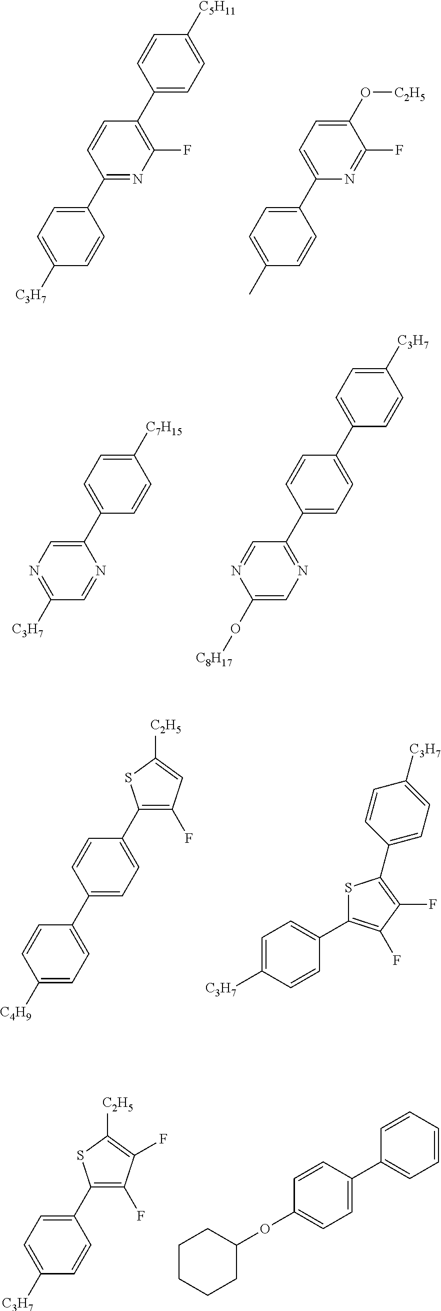

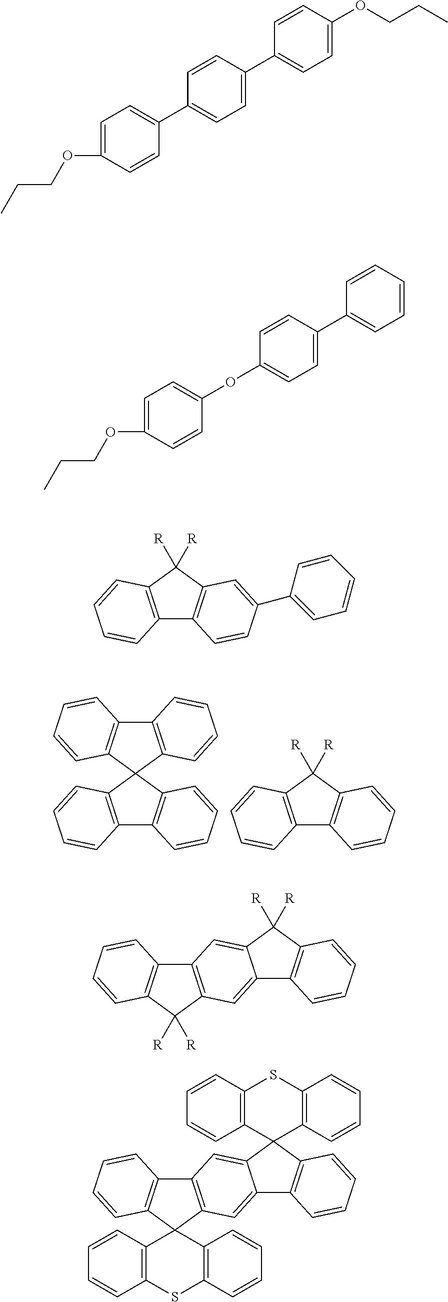

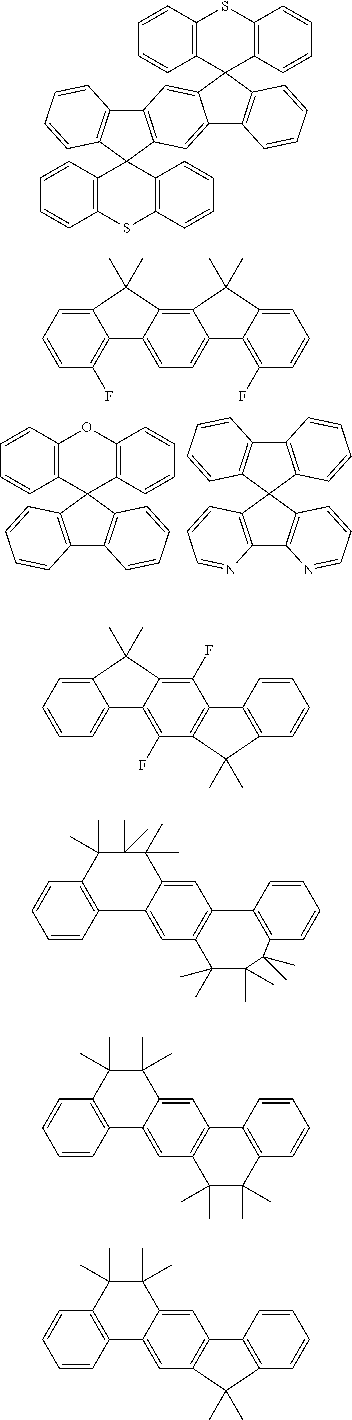

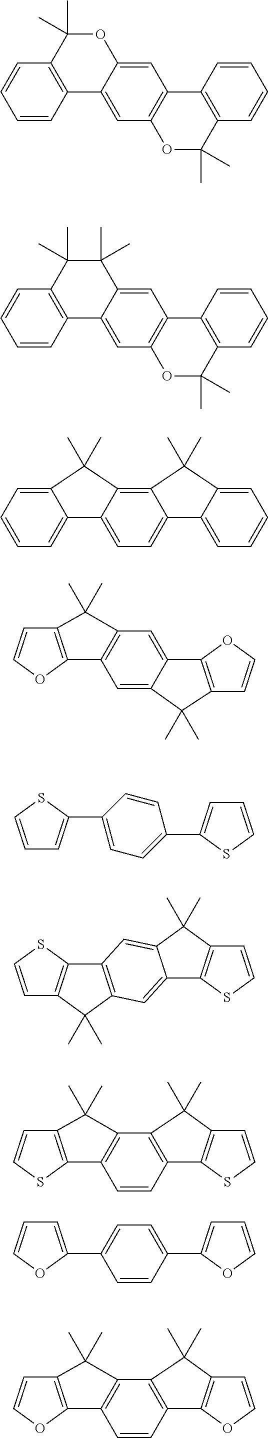

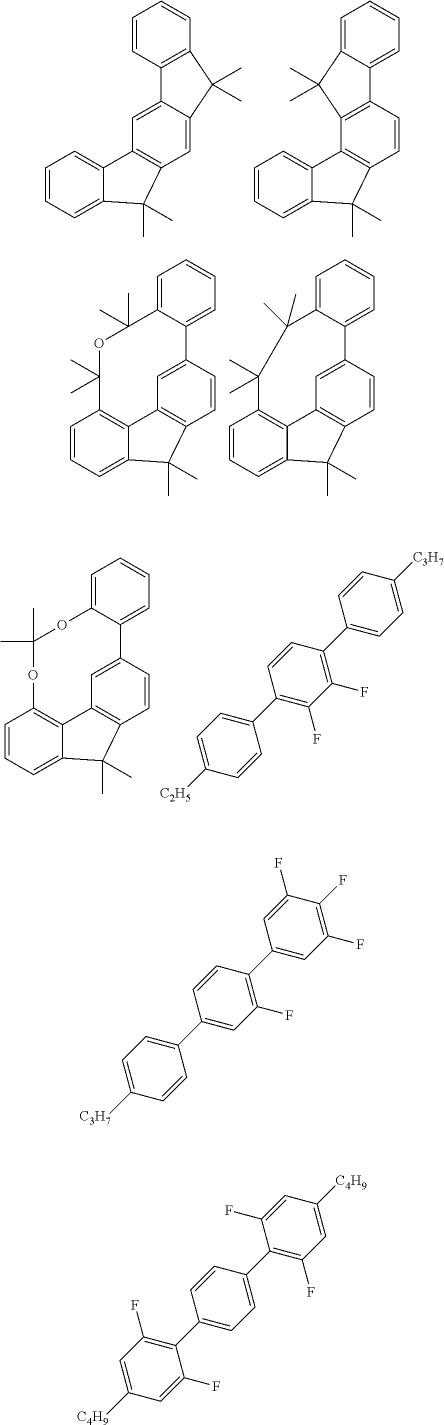

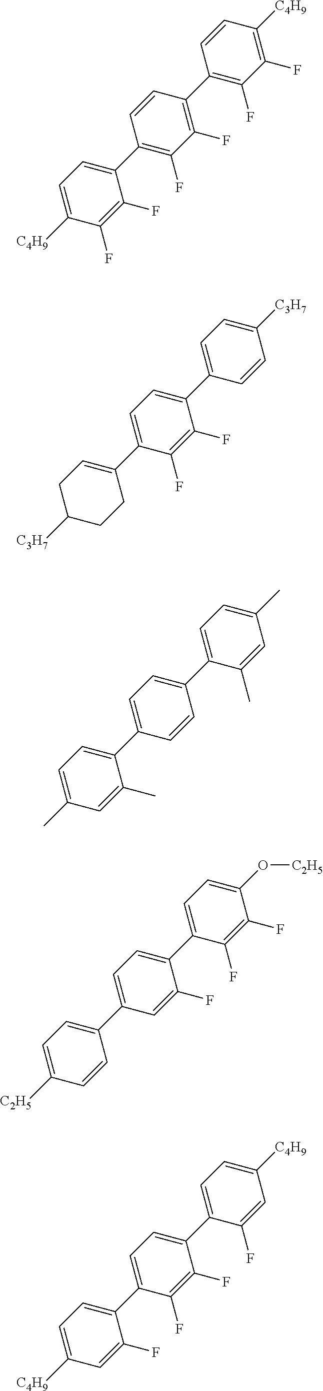

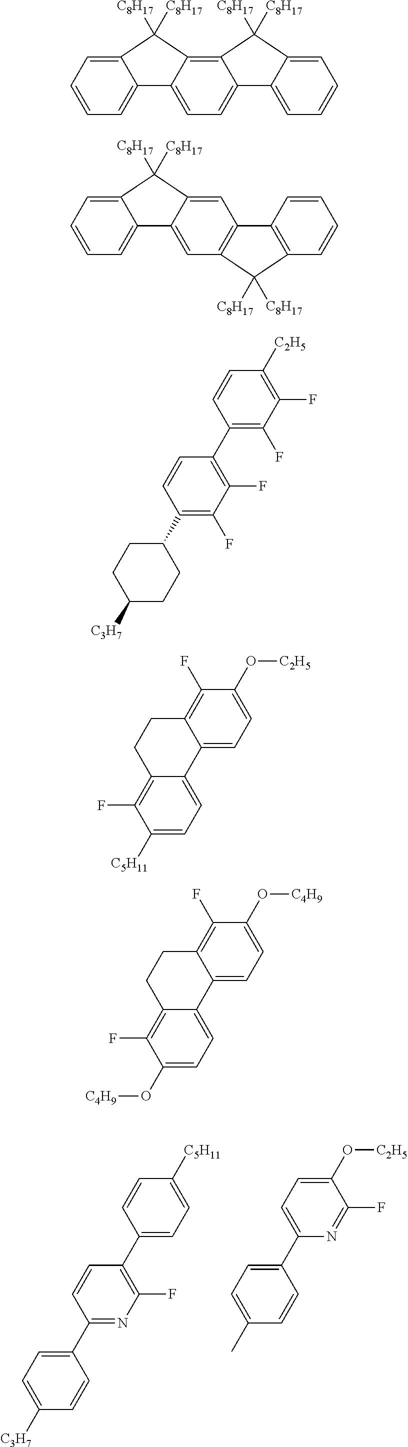

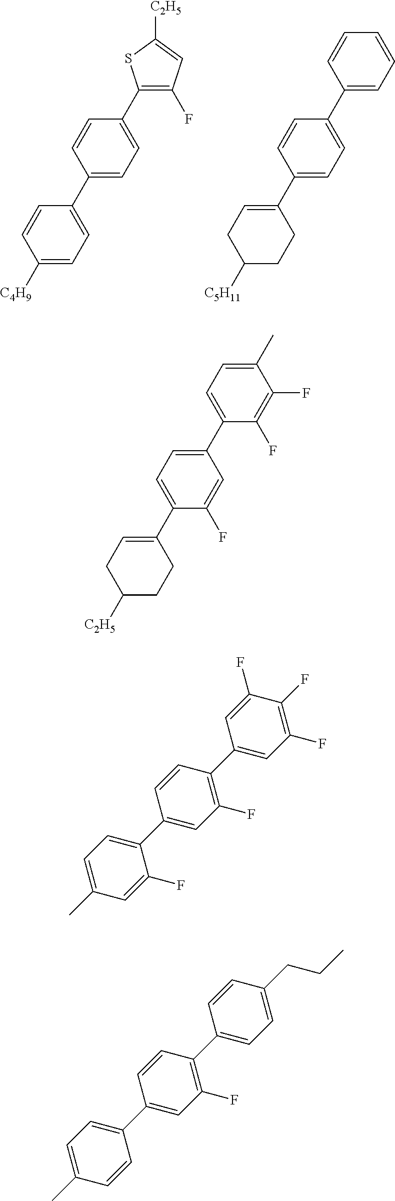

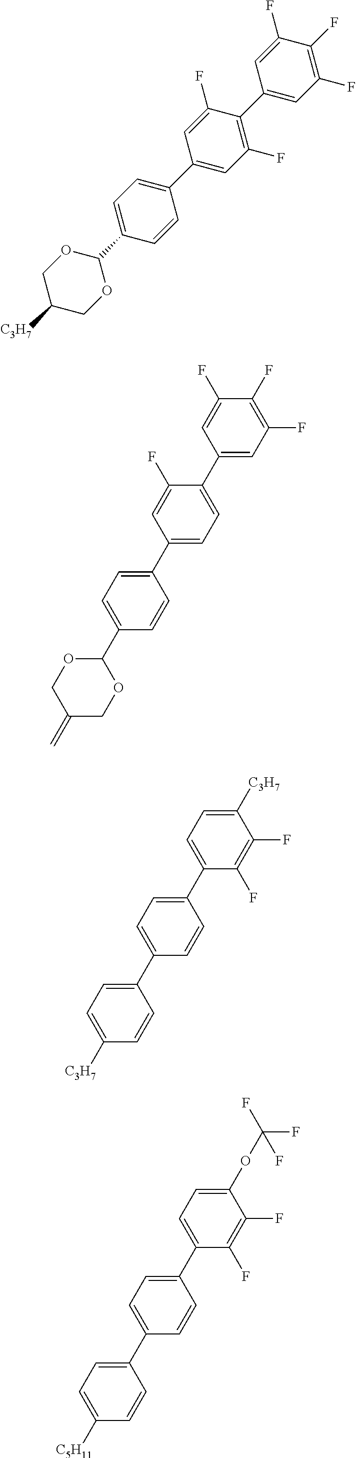

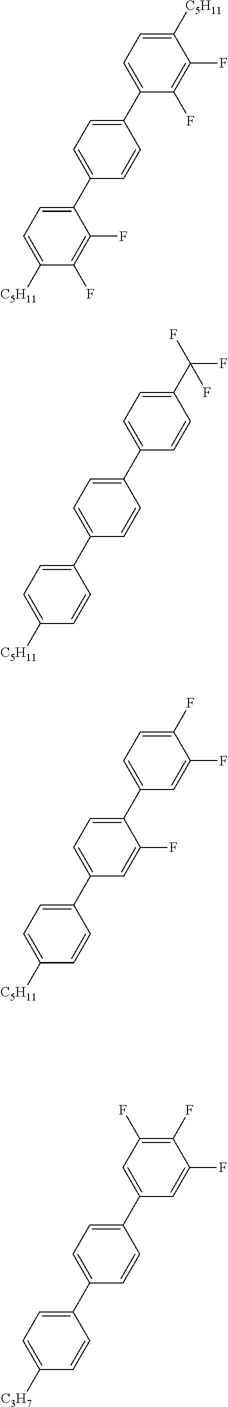

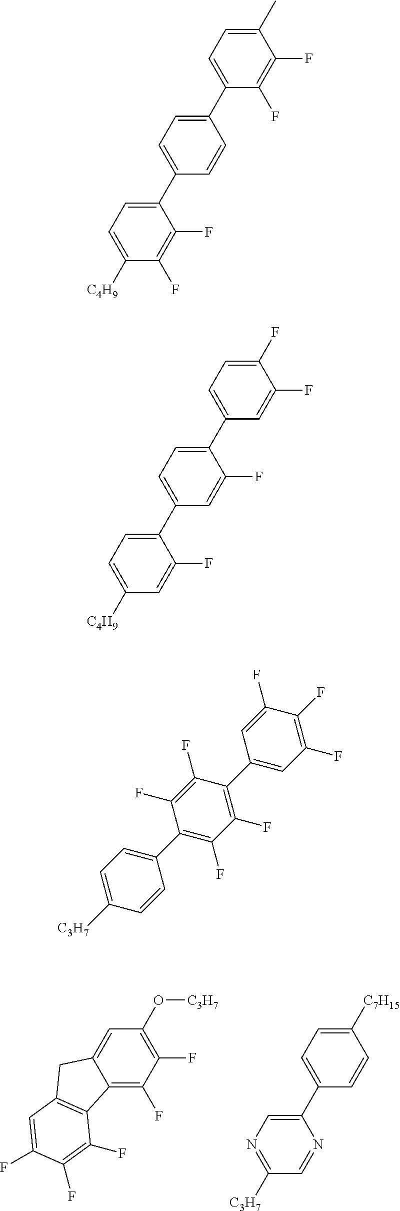

Further examples of preferred emitters which can be used in the emission layer are disclosed in EP 13000011.0 and in EP12008416.5

The emission layer of the device according to the invention furthermore preferably comprises at least one emitter of the following formulae (with reference) or derivatives thereof in the emission layer (EML):

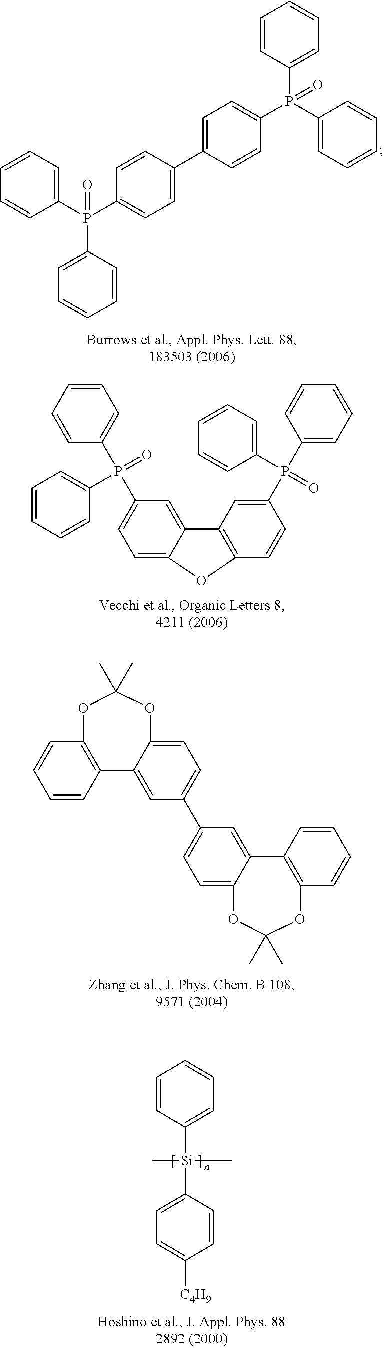

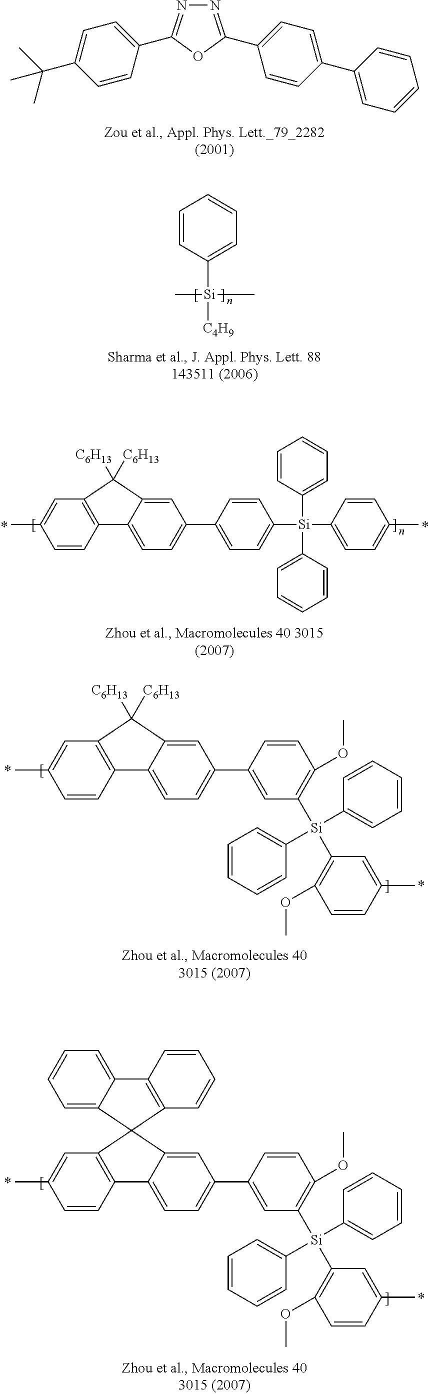

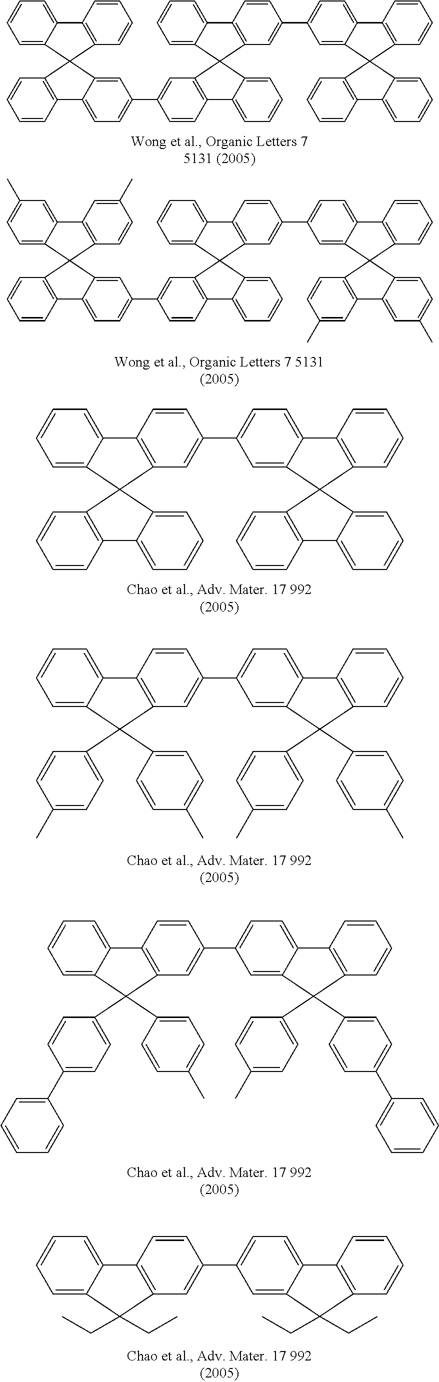

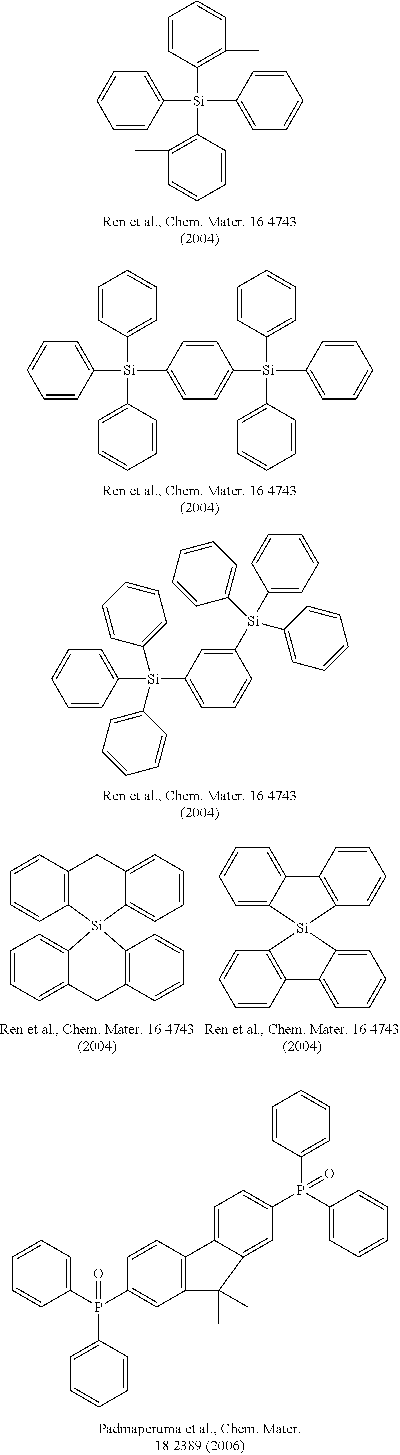

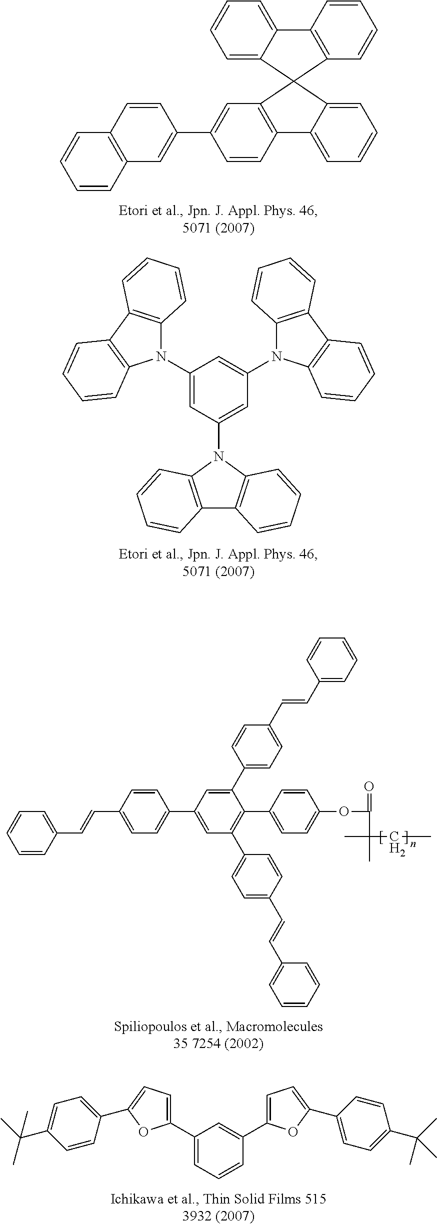

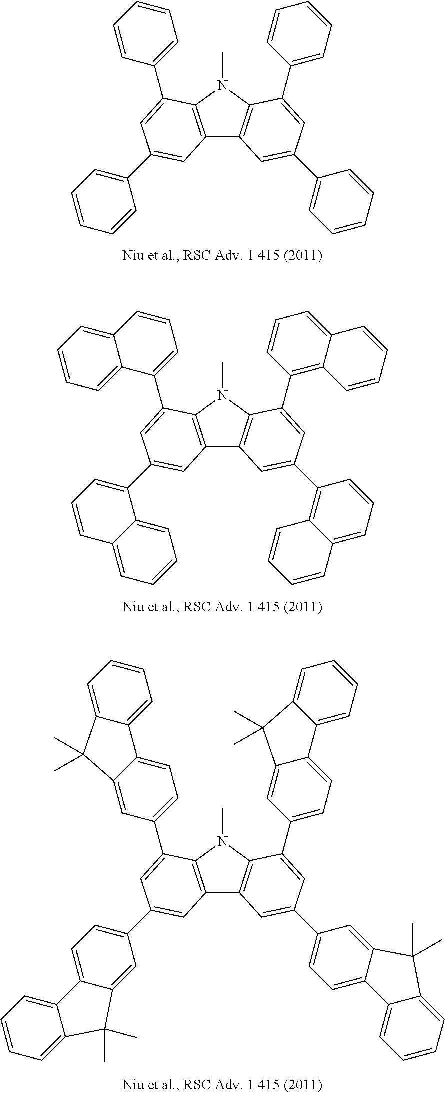 [SMILES: Cn1c2c(-c3ccccc3)cc(-c3ccccc3)cc2c2cc(-c3ccccc3)cc(-c3ccccc3)c21 ; Cn1c2c(-c3cccc4ccccc34)cc(-c3cccc4ccccc34)cc2c2cc(-c3cccc4ccccc34)cc(-c3cccc4ccccc34)c21 ; Cn1c2c(-c3ccc4c(c3)C(C)(C)c3ccccc3-4)cc(-c3ccc4c(c3)C(C)(C)c3ccccc3-4)cc2c2cc(-c3ccc4c(c3)C(C)(C)c3ccccc3-4)cc(-c3ccc4c(c3)C(C)(C)c3ccccc3-4)c21]

The organic electroluminescent device according to the invention may comprise further layers besides the layers already mentioned. Each layer that would be considered by the person skilled in the art in the area comes into question here. Particularly preferred layers which may occur in the according to the invention are selected from the group consisting of exciton-blocking layer (ExBL), electron-transport layer (ETL), electron-injection layer (ElL), electron-blocking layer (EBL), hole-transport layer (HTL), hole-injection layer (HIL), hole-blocking layer (HBL), and a further emission layer (EML).

Preferred organic electroluminescent devices in the sense of the present invention are organic light-emitting diodes (OLED), polymeric light-emitting diodes (PLED), organic light-emitting electrochemical cells (OLEC, LEC or LEEC).

In an especially preferred embodiment, the device according to the invention is an OLED, or OLEC or PLED.

The general structure and the functional principle of OLEDs is known to the person skilled in the art and disclosed, inter alia, in U.S. Pat. No. 4,539,507, U.S. Pat. No. 5,151,629, EP 0676461 and WO 1998/27136

The device is correspondingly (depending on the application) structured, provided with contacts and finally sealed, since the lifetime of the devices according to the invention is shortened in the presence of water and/or air.

In a preferred embodiment, the organic electroluminescent device according to the invention is characterised in that one or more layers are coated by means of a sublimation process, in which the materials are applied by vapour deposition in vacuum sublimation units at an initial pressure less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett, 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, ink-jet printing, gravure printing, dip coating, letterpress printing, doctor blade coating, roller printing, reverse-roller printing, web printing, spray coating, brush coating or pad printing, slot-die coating, but particularly preferably ink-jet printing or gravure printing. Soluble compounds are necessary for this purpose. High or improved solubility of the compounds to be applied can be achieved through suitable substitution of the compounds.

It is furthermore preferred, for the production of an organic electroluminescent device according to the invention, for one or more layers to be applied from solution and one or more layers to be applied by a sublimation process.

In a furthermore especially preferred embodiment, the device according to the invention is an OLEC (also called LEC or LEEC).

Organic light-emitting electrochemical cells (OLECs) comprise two electrodes, and a mixture or blend of electrolytes and an organic light-emitting species in between, as first disclosed by Pei & Heeger in Science (95), 269, pp1086-1088. The device according to the invention is therefore an OLEC, where at least one blocking layer, as described herein, is located between one of the electrodes and the emission layer (EML).

The exciton-blocking layer according to the invention can be applied by means of various methods, typically with the aid of “physical vapour deposition” (PVD), or “chemical vapour deposition” (CVD) or from solution.

Physical vapour deposition (PVD) encompasses a multiplicity of vacuum depositions and is a general term for describing various methods for the deposition of thin films on a substrate by condensation of an evaporated form of the desired material as film. Variants of PVD are, for example, e-beam, thermal vacuum evaporation, pulsed laser deposition and sputtering. The e-beam method is very preferred.

In a typical CVD process, the substrate is exposed to one or more volatile precursors which react and/or decompose on the substrate surface to give the desired compound. A number of CVD processes have been developed in the semiconductor industry, such as, for example, plasma-enhanced CVD, atomic layer deposition, metal organic chemical vapour deposition (MOCVD), vapour phase epitaxy (VPE) and direct liquid injection CVD (DLICVD).

In a very preferred embodiment, the blocking layer according to the invention is applied from solution (wet-chemical process). In accordance with the prior art, two wet-chemical processes are typically necessary for the application of inorganic thin layers to substrates:
1) by dispersion of inorganic nanoparticles. A disadvantage here is that it is very difficult to obtain very dense layers.
2) By “spray pyrolysis” (Reyna-Garcia, et al., J. Mater. Sci. 15, 439 (2004).) or sol-gel processes of a precursor of a corresponding inorganic compound, in particular metal oxides. The high temperatures often required and the need for water are disadvantageous.

This has the advantage over the prior-art process, i.e. the vapour deposition of nanoparticles, that the blocking layer is densified further, which results in a reliable organic light-emitting device having higher efficiency. The present invention therefore also relates to a process for the production of the device according to the invention, characterised in that the exciton-blocking layer is applied from solution comprising a precursor of an inorganic blocking material.

For the purposes of the present invention, preference is given to a process for the application of the exciton-blocking layer in which the exciton-blocking layer is applied from solution by dip coating, spin coating, ink-jet printing or flexographic/gravure printing, screen printing, nozzle printing or offset printing, letterpress printing, doctor blade coating, roller printing, reverse-roller printing, web printing, spray coating, brush coating or pad printing, slot-die coating.

Particular preference is given to a process for the application of the exciton-blocking layer, characterised in that
a. the inorganic material of the exciton-blocking layer is applied as precursor solution by dip coating, spin coating or ink-jet printing or flexographic/gravure printing and
b. subsequent precursor decomposition by heating the applied precursor layer and/or with the aid of UV radiation, which results in the inorganic exciton-blocking layer.

The heating is preferably carried out at temperatures of 100° C. or higher, preferably 150° C. or higher, very preferably 250° C. or higher

The heating or drying can also be carried out by irradiation with UV radiation of wavelength <400 nm.

Various materials can be employed as precursor. Examples of the use of a precursor material for MgO are described, for example, in Stryckmans et al. Thin Solid Films 1996, 283, 17 (from magnesium acetylacetonate above 260° C.) or Raj et al. Crystal Research and Technology 2007, 42, 867 (from magnesium acetate from 300° C. in several steps). An example of the use of a soluble ZrO₂ precursor material is described in Ismail et al. Powder Technology 1995, 85, 253 (from zirconium acetylacetonate via several steps between 200 to 600° C.). An example of the use of a soluble HfO₂ is known, for example, from Zherikova et al. Journal of Thermal Analysis and calorimetry 2008, 92, 729 (from hafnium acetylacetonate over several hours between T=150 and 500° C.). Beta-diketonates, such as the acetylacetonates of zirconium and hafnium, are employed in layer depositions from the gas phase by means of chemical vapour deposition (CVD).

It is preferred if the precursor contains at least one ligand from the class of the oximates besides a metal. It is particularly preferred in accordance with the invention if the ligands of the metal are a 2-(methoxyimino)alkanoate, 2-(ethoxyimino)alkanoate or 2-(hydroxyimino)alkanoate. The ligands are synthesised by condensation of alpha-keto acids or oxocarboxylic acids with hydroxylamines or alkylhydroxylamines in the presence of bases in aqueous or methanolic solution.

The precursors, for example hafnium or zirconium complexes, form at room temperature by reaction of an oxocarboxylic acid with at least one hydroxyl- or alkylhydroxylamine in the presence of a base, such as, for example, tetraethylammonium hydrogencarbonate or sodium hydrogencarbonate, and subsequent addition of an inorganic salt (for example hafnium or zirconium salt), such as, for example, zirconium oxochloride octahydrate and/or hafnium oxochloride octahydrate.

Alternatively, an oxcarboxylic acid can be reacted with a hydroxocarbonate of the metal (for example hafnium or zirconium) in the presence of at least one hydroxyl- or alkylhydroxylamine.

The oxocarboxylic acid employed can be all representatives of this class of compound. However, preference is given to the use of oxoacetic acid, oxo-propionic acid or oxobutyric acid.

The thermal conversion of the precursor into the functional oxide layers, such as, for example, hafnium oxide or zirconium oxide layer, is carried out at a temperature ≧100° C. The temperature is preferably between 150 and 200° C.

Further details and preferred embodiments of the process are revealed by the disclosure content of WO 2010/078907.

It is preferred if organometallic oximates are employed as precursor materials in the said process.

It is especially preferred if the exciton-blocking layer of the device according to the invention comprises HfO₂ or ZrO₂ or consists of one of the two materials.

In a furthermore very preferred embodiment, the present invention relates to a process for the production of the exciton-blocking layer in which the precursor materials used have the following structure

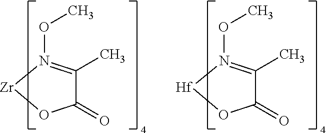

Organic electroluminescent devices which emit blue light and/or UV radiation can be employed in a versatile manner. Applications which require light or radiation having very short wavelengths and thus represent areas of application for the devices according to the invention are found, for example, in the area of life science and medicine (for example for cell imaging) or in the area of biosensors. The devices according to the invention are furthermore used in the electronics industry, solid-state lighting and for the curing of polymers and printing ink. The present invention therefore also relates to the use of the electroluminescent devices according to the invention in the said areas.

The devices according to the invention can also be employed for the light therapy (phototherapy) in humans and/or animal. The present invention therefore furthermore relates to the use of the devices according to the invention for the treatment, prophylaxis and diagnosis of diseases by means of phototherapy. The present invention still furthermore relates to the use, of the devices according to the invention for the treatment and prophylaxis of cosmetic conditions by means of phototherapy.

Phototherapy or light therapy is used in many areas of medicine and/or cosmetics. The devices according to the invention can therefore be employed for the therapy and/or prophylaxis and/or diagnosis of all diseases and/or in cosmetic applications for which the person skilled in the art considers using phototherapy. Besides irradiation, the term phototherapy also includes photo-dynamic therapy (PDT) as well as preservation, disinfection and sterilisation in general. It is not only humans or animals that can be treated by means of phototherapy or light therapy, but also any other type of living or non-living materials. These include, for example, fungi, bacteria, microbes, viruses, eukaryotes, prokaryotes, foods, drinks, water, drinking water, cutlery, medical/surgical instruments and equipment and other devices.

The term phototherapy also includes any type of combination of light therapy and other types of therapy, such as, for example, treatment with active compounds. Many light therapies have the aim of irradiating or treating exterior parts of living or non-living material, such as the skin of humans and animals, wounds, mucous membranes, the eye, hair, nails, the nail bed, gums and the tongue. In addition, the treatment or irradiation according to the invention can also be carried out inside an object in order, for example, to treat internal organs (heart, lung, etc.) or blood vessels or the breast.

The therapeutic and/or cosmetic areas of application according to the invention are preferably selected from the group of skin diseases and skin-associated diseases or changes or conditions, such as, for example, psoriasis, skin ageing, skin wrinkling, skin rejuvenation, enlarged skin pores, cellulite, oily/greasy skin, folliculitis, actinic keratosis, precancerous actinic keratosis, skin lesions, sun-damaged and sun-stressed skin, crows' feet, skin ulcers, acne, acne rosacea, scars caused by acne, acne bacteria, photomodulation of greasy/oily sebaceous glands and their surrounding tissue, jaundice, jaundice of the newborn, vitiligo, skin cancer, skin tumours, Crigler-Najjar, dermatitis, atopic dermatitis, diabetic skin ulcers, and desensitisation of the skin.

Particular preference is given for the purposes of the invention to the treatment and/or prophylaxis of psoriasis, acne, cellulite, skin wrinkling, skin ageing, jaundice and vitiligo.

Further areas of application according to the invention for the devices are selected from the group of inflammatory diseases, rheumatoid arthritis, pain therapy, treatment of wounds, neurological diseases and conditions, oedema, Paget's disease, primary and metastasising tumours, connective-tissue diseases or changes in the collagen, fibroblasts and cell level originating from fibroblasts in tissues of mammals, irradiation of the retina, neovascular and hypertrophic diseases, allergic reactions, irradiation of the respiratory tract, sweating, ocular neovascular diseases, viral infections, particularly infections caused by herpes simplex or HPV (human papillomaviruses) for the treatment of warts and genital warts.

Particular preference is given for the purposes of the invention to the treatment and/or prophylaxis of rheumatoid arthritis, viral infections and pain.

Further areas of application according to the invention for the devices are selected from winter depression, sleeping sickness, irradiation for improving the mood, the reduction in pain particularly muscular pain caused by, for example, tension or joint pain, elimination of the stiffness of joints and the whitening of the teeth (bleaching).

Further areas of application according to the invention for the devices are selected from the group of disinfections. The devices can be used for the treatment of any type of objects (non-living materials) or subjects (living materials such as, for example, humans and animals) for the purposes of disinfection, sterilisation or preservation. This includes, for example, the disinfection of wounds, the reduction in bacteria, the disinfection of surgical instruments or other articles, the disinfection or preservation of foods, of liquids, in particular water, drinking water and other drinks, the disinfection of mucous membranes and gums and teeth. Disinfection here is taken to mean the reduction in the living microbiological causative agents of undesired effects, such as bacteria and germs.

The devices according to the invention emit, so long as suitable emitters are used, in the UV and blue region of the spectrum. The precise wavelength can be adjusted towards longer wavelengths without difficulties by the person skilled in the art depending on the respective application.

In a particularly preferred embodiment of the present invention, the device is an organic light-emitting diode (OLED) or an organic light-emitting electro-chemical cell (OLEC) which are employed for the purposes of phototherapy. Both the OLED and the OLEC can have a planar or fibre-like structure having any desired cross section (for example round, oval, polygonal, square) with a single- or multilayered structure. These OLECs and/or OLEDs can be installed in other devices which comprise further mechanical, adhesive and/or electronic elements (for example battery and/or control unit for adjustment of the irradiation times, intensities and wavelengths). These devices comprising the OLECs and/or OLEDs according to the invention are preferably selected from the group comprising plasters, pads, tapes, bandages, sleeves, blankets, hoods, sleeping bags, textiles and stents.

The use of the said devices for the said therapeutic and/or cosmetic purpose is particularly advantageous compared with the prior art, since homogeneous irradiation in the high-energy blue region and/or in the UV region of lower irradiation intensities is possible at virtually any site and at any time of day with the aid of the devices according to the invention using the OLEDs and/or OLECs. The irradiation can be carried out as an inpatient, as an outpatient and/or by the patient themselves, i.e. without introduction and/or guidance by medical or cosmetic specialists. Thus, for example, plasters can be worn under clothing, so that irradiation is also possible during working hours, in leisure time or during sleep. Complex inpatient/outpatient treatments can in many cases be avoided or their frequency reduced. The devices according to the invention may be intended for re-use or be disposable articles, which can be disposed of after use once, twice or more times.

Further advantages over the prior art are, for example, lower evolution of heat and emotional aspects. Thus, newborn being treated owing to jaundice typically have to be irradiated blindfolded in an incubator without physical contact with the parents, which represents an emotional stress situation for parents and newborn. With the aid of a blanket according to the invention comprising the OLEDs and/or OLECs according to the invention, the emotional stress can be reduced significantly. In addition, better temperature control of the child is possible due to reduced heat production of the devices according to the invention compared with conventional irradiation equipment.

The present invention therefore also relates, in particular, to the device according to the invention for use in medicine for phototherapy.

The present invention also relates to the device according to the invention for use for the treatment of the skin by means of phototherapy.

The present invention also relates to the device according to the invention for use for the treatment of psoriasis by means of phototherapy.

The present invention also relates to the device according to the invention for use for the treatment of jaundice by means of phototherapy.

The present invention also relates to the device according to the invention for use for the treatment of jaundice of the newborn by means of phototherapy.

The present invention also relates to the device according to the invention for use for the treatment of acne by means of phototherapy.

The present invention also relates to the device according to the invention for use for the treatment of inflammation by means of phototherapy.

The present invention also relates to the device according to the invention for use for the treatment of atopic eczema by means of phototherapy.

The present invention also relates to the device according to the invention for use for the treatment of skin ageing by means of phototherapy

The present invention furthermore relates to the use of the devices according to the invention in the cosmetics area for phototherapy.

In particular, the present invention relates to the use of the devices according to the invention for the phototherapeutic reduction and/or for the photo-therapeutic prevention of the formation of skin wrinkles and skin ageing.

The present invention also relates to a method for the cosmetic treatment of the skin by phototherapy using a device according to the invention.

Examples of cosmetic areas of application of the phototherapy according to the invention are acne, skin ageing, crows' feet, skin wrinkling and cellulitis, to mention but a few.

The devices according to the invention and the process according to the invention are distinguished by the following surprising advantages over the prior art:
The devices according to the invention have improved efficiencies, higher radiation intensities.
2. The devices according to the invention are easy to produce and are suitable for an economical mass-production process.
3. The compounds according to the invention, employed in organic electroluminescent devices, result in high efficiencies and in steep current/voltage curves with low use voltages.

These above-mentioned advantages are not accompanied by an impairment in the other electronic properties.

It should be pointed out that variations of the embodiments described in the present invention fall within the scope of this invention. Each feature disclosed in the present invention can, unless explicitly excluded, be replaced by alternative features which serve the same, an equivalent or a similar purpose. Thus, each feature disclosed in the present invention should, unless stated otherwise, be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, should be regarded as inventive themselves and not merely as part of the embodiments of the present invention. Independent protection may be granted for these features in addition or as an alternative to each invention claimed at present.

The teaching regarding technical action disclosed with the present invention can be abstracted and combined with other examples.

The invention is explained in greater detail by the following examples and drawings without wishing to restrict it thereby.

### BRIEF DESCRIPTION OF THE FIGURES:

FIG. 1 shows the electroluminescence spectra of OLED1 and OLED-Ref1.

FIG. 2 shows the electroluminescence spectra of OLED2, OLED3 and OLED-Ref2.

### EXAMPLES

### Example 1

### Materials

The two following emitters E1 and E2 are used.

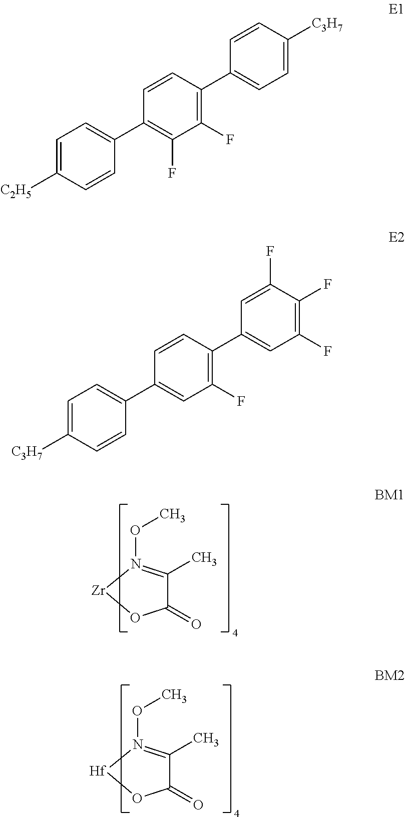

The synthesis of E1 is disclosed in EP 329752, that of E2 is disclosed in EP 440082.

The host used is polystyrene (PS) from Fluka (81414, Mw 500000, Mn 490000)). BM1 and BM2 are precursor materials for ZrOₓ and HfOₓ respectively (x≦2)). The synthesis of the precursor is carried out in accordance with WO 2010/078907.

### Example 2

### Solutions and Compositions Comprising Matrix Materials and Emitters

Solutions as summarised in Table 1 are prepared as follows: firstly, the mixtures of host and emitter are dissolved in 10 ml of toluene and stirred until the solution is clear. The solution is filtered using a Millipore Millex LS, hydrophobic PTFE 5.0 μm filter.

TABLE 1

| | Compo- | Ratio (based | Concen- |
|---|---|---|---|
| | sition | on weight) | tration |
| Solution 1 | PS:E1 | 70%:30% | 16 mg/ml |
| Solution 2 | PS:E2 | 70%:30% | 16 mg/ml |

The solutions are used for the production of the emitting layer of OLEDs. The corresponding solid composition can be obtained by evaporating the solvent from the solutions. This can be used for the preparation of further formulations.

### Example 3

### Production of the Organic Electroluminescent Devices

OLEDs Ref1 and Ref2 have the following structure: ITO/PEDOT/EML/cathode. The two OLEDs serve as reference examples. They are produced using the corresponding solutions from Table 1 in accordance with the following procedure:
1. Application of 80 nm of PEDOT (Clevios™ P VP Al 4083) to an ITO-coated glass substrate by spin coating. Subsequent drying by heating at 120° C. in a clean room for 10 min.
2. Application of a 100 nm emitting layer by spin coating a solution in accordance with Table 1 in an Ar glove box.
3. Drying of the device by heating at 180° C. for 10 min.
4. Application of a Ba/Al cathode by vapour deposition (3 nm+150 nm)
5. Encapsulation of the device

OLED1-OLED3 are devices according to the invention having the following structure: ITO/BL/EML/cathode, where BL stands for the exciton-blocking layer. They are, are produced using the solutions from Table 1 by the process described for OLED-Ref1 and OLED-Ref2, where step 1 is replaced by the following coating procedure.
1. Coating of less than 5 nm of BL onto an ITO-coated glass substrate by
   a. spin coating of a 2.6 wt % solution of BM1 or BM2 in 2-ethoxyethanol in a clean room;
   b. subsequent drying by heating at 450° C. in a clean room for 10 min.;
   c. subsequent treatment with UV ozone at 5 min.

The OLEDs are summarised in Table 2.

TABLE 2

| Device | BL | EML |
|---|---|---|
| Ref1 | PEDOT | PS(70%):E1(30%) |
| Ref2 | PEDOT | PS(70%):E2(30%) |
| OLED1 | HfOₓ (from BM2) | PS(70%):E1(30%) |
| OLED2 | ZrOₓ (from BM1) | PS(70%):E2(30%) |
| OLED3 | HfOₓ (from BM2) | PS(70%):E2(30%) |

### Example 4

### Characterisation of the OLEDs

Firstly, electroluminescence spectra (EL) of the OLEDs obtained in this way are measured. The EL spectra were measured by means of Ocean Optics UBS2000 at 30 V.

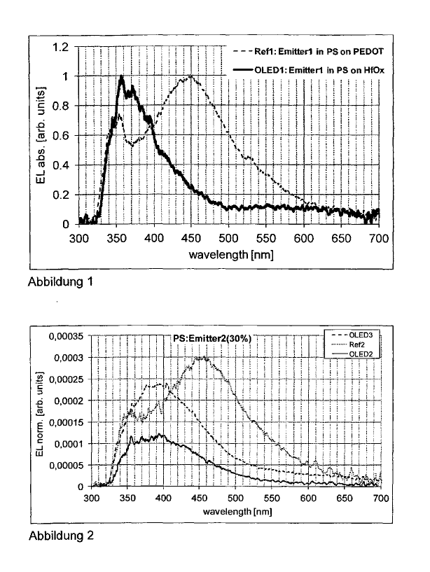

The EL spectra of the devices comprising E1 are summarised in FIG. 1. OLED1, with HfOₓ as BL, exhibits significantly higher intensities at short wavelengths, i.e. in the UV region of the spectrum, than Ref1. Furthermore, the UV spectrum of OLED1 is significantly purer than that of Ref1. The EL spectrum of OLED1 also has a blue component, which is possibly attributable to an interface effect between PEDOT and the emitting layer.

The EL spectra with Emitter2 are summarised in FIG. 2. OLED2 and OLED3, with ZrOₓ and HfOₓ as BL, exhibit improved UV spectra than Ref2.

Further optimisations can be achieved by means of various possibilities on the basis of the present technical teaching according to the invention. To this end, the person skilled in the art will be able to carry out a number of routine experiments and achieve these improvements without inventive step. Thus, for example, the use of a co-matrix or the use of a substrate which exhibits higher UV transparency will result in improvements in the performance data of the devices according to the invention.

1-19. (canceled)

20. Organic electroluminescent device comprising an anode, a cathode, an emission layer and at least one exciton-blocking layer between emission layer and anode, wherein the exciton-blocking layer comprises an inorganic material.

21. The device according to claim 20, wherein the inorganic material in the exciton-blocking layer has a band gap of at least 3.5 eV.

22. The device according to claim 20, wherein the inorganic material is selected from a metal chalcogenide.

23. The device according to claim 20, wherein the blocking layer comprises a metal oxide selected from BaO, MgO, SrO, HfO₂, ZrO₂, GeO₂, Ga₂O₃, Ta₂O₅, or any one mixture of the selected metal oxides.

24. The device according to claim 20, wherein the blocking layer comprises HfO₂, ZrO₂, or a mixture thereof.

25. The device according to claim 20, wherein the device emits radiation in the range from 200 to 380 nm and preferably in the range from 280 to 360 nm.

26. The device according to claim 20, wherein the emission layer comprises at least one emitter that emits light at a wavelength in the range from 200 to 380 nm.

27. The device according to claim 20, wherein the emission layer comprises, as emitter or host, at least one compound of the general formula (1). 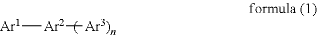
and where the following applies to the symbols and indices used:
Ar¹, Ar² and Ar³
are, identically or differently, five- or six-membered aromatic or heteroaromatic rings, optionally substituted by one or more radicals R¹, which may be independent of one another;
n is 0 or 1;
X is on each occurrence, identically or differently, CR¹ or N;
Q is on each occurrence, identically or differently, X=X, NR¹, O, S, or Se.
R¹ is, identically or differently on each occurrence, H, D, F, Cl, N(R²)₂, CN, Si(R²)₃, B(OR²)₂, C(═O)R², P(═O)(R²)₂, P(R²)₂, S(═O)R², a straight-chain alkyl, alkoxy or thioalkoxy group having 1 to 40 C atoms or a branched or cyclic alkyl, alkoxy, alkylalkoxy or thioalkoxy group having 3 to 40 C atoms, each of which may be substituted by one or more radicals R², where one or more non-adjacent CH₂ groups which is not bonded directly to the ring of the formula (1) may be replaced by R²C═CR², C≡C, Si(R²)₂, Ge(R²)₂, Sn(R²)₂, C═O, C═S, C═Se, C═NR², P(═O)(R²), SO, SO₂, NR², O, S or CONR² and where one or more H atoms may be replaced by D, F, Cl, or CN, or an aromatic or heteroaromatic ring having 5 to 18 aromatic ring atoms, which may in each case be substituted by one or more radicals R², or an aryloxy, arylalkoxy or heteroaryloxy group having 5 to 60 aromatic ring atoms, which may be substituted by one or more radicals R², or two or more substituents R¹ can combine to form a non-aromatic ring system;
R² is, identically or differently on each occurrence, H, D, F, Cl, N(R³)₂, CN, Si(R³)₃, B(OR³)₂, C(═O)R³, P(═O)(R³)₂, S(═O)R³, a straight-chain alkyl, alkoxy or thioalkoxy group having 1 to 40 C atoms or a straight-chain alkenyl or alkynyl group having 2 to 40 C atoms or a branched or cyclic alkyl, alkenyl, alkynyl, alkoxy, alkylalkoxy or thioalkoxy group having 3 to 40 C atoms, each of which may be substituted by one or more radicals R³, where one or more non-adjacent CH₂ groups may be replaced by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³ and where one or more H atoms may be replaced by D, F, Cl or CN, or an aromatic or heteroaromatic ring having 5 to 18 aromatic ring atoms, which may in each case be substituted by one or more radicals R³, or two or more substituents R¹ can combine to form a non-aromatic ring system;
R³ is, identically or differently on each occurrence, H, D, F or an aliphatic, aromatic and/or heteroaromatic hydrocarbon radical having 1 to 18 C atoms, wherein one or more H atoms is oprionally replaced by F; or two or more substituents R³ can combine to form a non-aromatic mono- or polycyclic, aliphatic ring system;
with the proviso that the compound of the formula (1) does not contain any condensed aromatic or heteroaromatic ring systems, and the number of π-electrons in a fully conjugated moiety is not more than 18.

28. The device according to claim 20, further comprising layers selected from the group consisting of exciton-blocking layer (ExBL), electron-transport layer (ETL), electron-injection layer (EIL) and a further emission layer (EML).

29. The device according to claim 20, wherein the device is selected from an organic light-emitting diode (OLED), polymeric light-emitting diode (PLED), or organic light-emitting electrochemical cell (OLEC, LEC or LEEC)

30. A process for making the electroluminescent device according to claim 20, comprising applying the exciton-blocking layer by physical vapour deposition or by solution.

31. The process according to claim 30, wherein the exciton-blocking layer is applied by the solution with an application process selected from screen printing, flexographic printing, nozzle printing, offset printing, ink jet printing, gravure printing, dip coating, spin coating, letterpress printing, doctor blade coating, roller printing, reverse-roller printing, web printing, spray coating, brush coating, pad printing, or slot-die coating.

32. The process according to claim 30, wherein the exciton-blocking layer is applied by the solution with an application process selected from dip coating, spin coating, ink-jet printing or flexographic/gravure printing, and
heating or drying of the applied solution layer to provide the inorganic exciton-blocking layer.

33. The process according to claim 32, wherein the inorganic material of the exciton-blocking layer is applied as an organometallic oximate precursor.

34. The process according to claim 30, wherein the inorganic material of the exciton-blocking layer is HfOₓ or ZrOₓ, where x≦2.

35. The process according to claim 34, wherein organometallic oximate precursor is selected from the zirconium oximate or hafium oximate complexes as

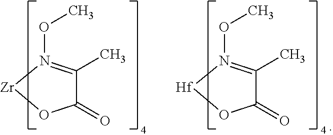

36. The device according to claim 20, wherein the inorganic material in the exciton-blocking layer has a band gap of at least 4.0 eV.

37. The device according to claim 27, wherein the emission layer comprises at least one emitter that emits light at a wavelength in the range from 280 to 360 nm.

38. A process of treating a patient with phototherapy comprising irradiating a patient with light obtained from the device according to claim 20.

39. The process according to claim 38 wherein the skin of the patient is irradiated.

40. A medical treatment comprising irradiating skin of a patient diagnosed with psoriasis with light obtained from the device according to claim 20.
